(19) United States
(12) Patent Application Publication
(10) Pub. No.: US 2006/0047248 A1
(43) Pub. Date: Mar. 2, 2006

(54) Catheter loop manager

(76) Inventors: Andrew L Poutiatine, San Anselmo, CA (US); Edward M Gillis, San Jose, CA (US); Riten Parikh, San Jose, CA (US); James A Filice, Los Gatos, CA (US)

Correspondence Address: DURECT CORPORATION, 10240 BUBB ROAD, CUPERTINO, CA, 95014, US

(21) Appl. No.: 10/537,196

(22) PCT Filed: Dec. 4, 2003
(86) PCT No.: PCT/US03/38548

§ 371 Date: Jun. 2, 2005

Related U.S. Application Data
(60) Provisional application No. 60/430,759, filed on Dec. 4, 2002.

(51) Int. Cl. A61M 5/32

(52) U.S. Cl. 604/174

(57) ABSTRACT

A catheter loop manager apparatus which controls the slack portion of a catheter located adjacent to an implanted pump and prevents fibrotic encapsulation of the slack catheter portion. The catheter loop manager comprises a retainer element configured to associate with an implantable pump and to releasibly hold or retain a loop or section of catheter adjacent to the pump. A pull tool is used to releasibly engage a catheter section and pull the catheter section into a loop within a retention channel in retainer element.

### FIELD OF THE INVENTION

This invention pertains generally to implantable drug delivery systems, and more particularly to a catheter loop manager which controls the excess catheter at the pump end to prevent fibrotic encapsulation of the catheter slack and avoid damage to the catheter during pump replacement procedures.

### BACKGROUND OF THE INVENTION

Implantable drug delivery devices are increasingly used as therapeutic tools for treatment of a variety of conditions and diseases, especially where a prolonged period of therapy is required. Implantable drug delivery devices avoid patient inconvenience and discomfort associated with administration of multiple doses of an agent, and further provide for enhanced therapeutic benefits due to avoidance of bolus doses, improved patient compliance with dosage regimens, and providing generally a constant blood serum level of delivered drugs.

Various implantable drug delivery systems have been developed using different technologies to accomplish movement of drug (typically in a drug formulation, e.g., a solvent) from within a reservoir in the device through an exit port or orifice to a treatment site in the subject. These delivery technologies have been based on, inter alia, diffusive, erodible, and convective mechanisms. Exemplary delivery systems employing convection include, but are not limited to, electromechanical pumps, osmotic pumps, electro-osmotic pumps, electro-chemical pumps, hydrolytic systems, piezoelectric pumps, elastomeric pumps, vapor pressure pumps, and electrolytic pumps.

The location of a specific drug delivery site in the body often will be non-optimal for locating an implantable pump. This situation occurs, for example, in the delivery of hydromorphone to the spinal column. In such instances the pump is implanted remotely from the actual drug delivery site and the is drug transported from the pump to the delivery site via an implanted catheter. Generally, an excess length of catheter tubing is required adjacent the pump implant incision to provide sufficient slack to allow external assembly of the pump and catheter end prior to implanting. When the pump is inserted into the implant incision, the excess portion of catheter will form a slack loop adjacent to the pump.

The presence of an uncontrolled, slack, excess portion of catheter within the implant incision creates some important problems. The uncontrolled portion of catheter may kink or otherwise adopt a configuration which hinders drug delivery after the implant incision is closed and sutured. The slack portion of catheter will ultimately become fibrotically encapsulated in the tissue adjacent to the pump. The internal drug reservoir of the implanted pump will become depleted over time and require replacement, and the fibrotically encapsulated catheter greatly complicates pump replacement. Since the location of the slack portion of catheter is not predictable, the catheter may be inadvertently cut or otherwise damaged by a scalpel during an incision to replace the pump. The fibrotic encapsulation of the slack portion of catheter makes it difficult to remove the pump from the incision without damaging the catheter at the pump connection, and careful surgical excision is often necessary to free the spent pump. Damage to the catheter end will typically require re-implantation of the entire catheter at substantial cost, inconvenience and patient discomfort.

There is accordingly a need for a catheter loop manager which controls the excess or slack portion of catheter associated with an implanted pump, which holds the slack portion in a precisely controlled loop adjacent to the pump, which avoids fibrotic encapsulation of slack catheter portions, and which generally avoids surgical complications associated with have heretofore interfered with replacement of implantable pumps. The present invention satisfies these needs, as well as others, and generally overcomes the deficiencies found in the background art.

### SUMMARY OF THE INVENTION

The present invention is a catheter loop manager apparatus which controls the slack portion of a catheter located adjacent to an implanted pump and prevents fibrotic encapsulation of the slack catheter portion. In general terms, the catheter loop manager comprises a retainer element configured to associate with an implantable pump and to releasibly hold or retain a loop or section of catheter adjacent to the pump. More preferably, the retainer element couples to the implantable pump and retains a loop of catheter in close proximity to the pump at a known location, preferably within a channel in the retainer element. The catheter loop manager also preferably comprises a pull tool configured to releasibly engage a catheter section and pull the catheter section into a loop within the retainer element.

By way of example, and not necessarily of limitation, the retainer element includes an elongated, tubular retainer body with an external surface structured and configured to conform to the outer surface of an implantable pump. A tip portion is joined to the first or front end of the retainer body and includes a bore configured to receive a connector ferrule on the pump. The bore in the tip portion allows a catheter end to couple to the ferrule while the retainer element is mounted on the implantable pump. The retainer body and tip portion are preferably integral to each other, with the retainer element being fabricated from a single piece of resilient, elastomeric polymeric material such as a silicone rubber. One or more lateral grooves may be included on the exterior surface of the tip portion and/or retainer body to accommodate sutures and facilitate the suturing of the loop manager and pump within an implant incision.

An elongated channel extends through the retainer body and communicates with the front and back ends thereof. When the retainer element is joined to the pump, the channel is substantially aligned with the longitudinal axis of the pump. The elongated channel in the retainer body and the bore in the tip portion preferably are substantially parallel in orientation with respect to each other. The channel is structured and configured to slidably receive the pull tool and to retain an elongated loop of catheter therewithin. The pull tool includes a hook adjacent a first end which is configured to fit through the elongated channel and releasibly engage a catheter. The pull tool also preferably includes a handle portion at a second end to facilitate manipulation.

During pump implant procedures, a slack or excess “service” portion of catheter is generally provided adjacent the pump implant incision to allow manipulation of the pump while it is attached to the catheter. The catheter loop manager of the invention advantageously provides control of the slack portion of catheter and avoids damage thereto by holding the excess catheter portion as a catheter loop within the channel of the retainer body.

In operation, the retainer element is coupled to a pump prior to implanting. The catheter end is passed through the bore in the tip portion and affixed to the connector ferrule. The connector ferrule on the end of the pump fits into the bore in the tip section, and the arcuate surface on the retainer body is positioned adjacent to the cylindrical body of the pump. The hook of the guide tool is then passed through the channel in the retainer body and is used to hook or otherwise engage the slack portion of the catheter and draw the catheter portion into the channel of the retainer body. As the slack portion of catheter is drawn into the retainer body it forms a well defined loop within the channel which will provide a “service loop” for subsequent pump replacement procedures. The hook on the pull tool is then disengaged, and the pump and attached retainer element with the internal catheter loop are positioned within the pump incision and sutured into place using the lateral grooves on the retainer element, after which the incision is closed. The catheter loop is retained within the channel for the duration of the implant.

When the drug reservoir internal to the implanted pump has become depleted, a new incision is made to remove the depleted pump. Since the slack portion of catheter is retained or “managed” within the retainer element, there is no risk of cutting the catheter portion. The sutures holding the pump and retainer element in place are removed, and the pump and attached retainer element are withdrawn from the incision. The loop of catheter is withdrawn from the channel in the retainer body as the pump and retainer element are removed from the incision to again provide a service portion of catheter to facilitate handling of the pump prior to detachment of the catheter, and the attachment of a new pump. Since the excess catheter portion has been retained within the channel of the retainer body for the duration of the implant, the excess catheter portion is free from any fibrotic inclusion by surrounding tissue which would otherwise have occurred. The retainer element and exhausted pump may be disposed of, or the retainer element may be detached from the spent pump, sterilized, and re-used.

The invention thus provides a method for managing a service loop or portion of catheter associated with an implant incision which comprises providing a retainer element in association with an implantable pump, affixing a catheter end to a connector on the pump, and drawing an excess portion of catheter into the retainer element to form a catheter loop therein. The method also preferably comprises withdrawing the catheter loop from the retainer element, after depletion of the pump, to form a service portion of catheter. The method further preferably comprises coupling the retainer element to the pump, and suturing the retainer element and pump within the implant incision.

An object of the invention is to provide a catheter loop manager which retains a service portion of catheter as a loop within a retainer element associated with an implanted pump.

Another object of the invention is to provide a catheter loop manager which prevents the service portion of a catheter from becoming fibrotically enclosed within tissue during the implant period of an implantable pump.

Another object of the invention is to provide a catheter loop manager which prevents inadvertent damage to the service portion of a catheter during implantable pump replacement procedures.

Another object of the invention is to provide a catheter loop manager that avoids kinking or restriction of a catheter associated with an implanted pump which would interfere with drug delivery from the pump through the catheter.

Another object of the invention is to provide a catheter loop manager which facilitates the surgical procedures associated with implanting and interchanging of implantable pumps.

Further objects and advantages of the invention will be brought out in the following portions of the specification, wherein the detailed description is for the purpose of fully disclosing the preferred embodiment of the invention without placing limitations thereon.

### BRIEF DESCRIPTIONS OF THE DRAWINGS

The present invention will be more fully understood by reference to the following drawings, which are for illustrative purposes only.

FIG. 1 is a perspective view of a catheter loop manager apparatus in accordance with the present invention shown together with an implantable pump and a section of catheter.

FIG. 2 is an exploded view of the catheter loop manager apparatus and assembled implantable pump and catheter of FIG. 1.

FIG. 3A through FIG. 3D illustrate the use of the catheter loop manager apparatus of the invention, with the retainer element shown in cross-section.

### DETAILED DESCRIPTION OF THE INVENTION

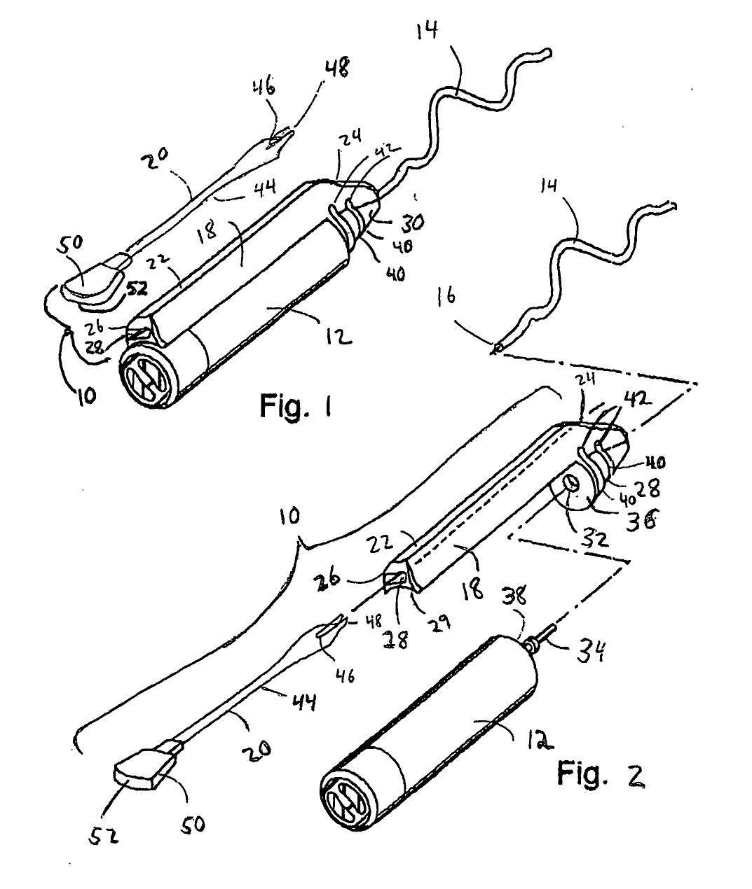

Referring more specifically to the drawings, for illustrative purposes the present invention is embodied in the apparatus and method shown generally in FIG. 1 through FIG. 3. It will be appreciated that the apparatus may vary as to configuration and as to details of the parts, and that the method may vary as to details and the order of the steps, without departing from the basic concepts as disclosed herein. The invention is disclosed generally in terms of use with an implantable pump for drug delivery applications. However, it will be readily apparent to those skilled in the art that the invention may be used to control the end portion of an implanted catheter in various situations where management of the catheter end is desirable, and the details and specificities discloses herein are only exemplary and should not be considered limiting.

Referring now to FIG. 1 through FIG. 3, a catheter loop manager apparatus 10 in accordance with the present invention is shown together with an implantable pump 12 and a section or portion of catheter 14 having an end 16. The catheter loop manager apparatus 10 comprises a retainer element 18 configured to couple to or otherwise associate with implantable pump 12, and a pull tool 20 used with retainer element 18 as described in more detail below. Implantable pump 12 may be any type of pump including, for example, mechanical, electrical, osmotic, diffusion, electrochemical etc. The catheter loop manager 10 of the invention may, however, be used with any type of implantable pump.

Retainer element 18 preferably includes an elongated, tubular retainer body 22 with first and second ends 24, 26. A longitudinal channel 28 extends through retainer body 22 and communicates with ends 24, 26. Retainer body 18 preferably has an elongated arcuate surface 29 which is structured and configured to conform to the outer surface of implantable pump 12, with retainer body 22 and channel 28 being oriented in a direction substantially parallel to the longitudinal axis (not shown) of pump 12 when retainer element 18 is coupled to pump 12.

Retainer element 18 also preferably includes a tip portion 30 joined to first end 24 of retainer body 22. Tip portion 30 includes a bore 32 extending therethrough which is structured and configured to accommodate a connector ferrule 34 on pump 12. Bore 32 is also structured and configured to allow catheter end 16 to pass through bore 32 and join to connector 34. Tip portion as shown is tapered or cone-shaped, and includes a generally flat back surface 36 which conforms in shape to the top end 38 of pump 12. The channel 28 in retainer body 22 and bore 32 in tip section preferably are substantially parallel to each other in orientation.

Retainer body 22 and tip portion 30 preferably are integral to each other, with retainer element 18 being fabricated from a single molded piece of resilient or flexible polymeric material such as silicone rubber or like material. Preferably, one or more grooves 40 extend laterally around tip portion 30 and communicate with corresponding holes 42 which extend laterally through retainer body 22. Grooves 40 and holes 42 accommodate sutures (not shown) that hold retainer element 18 and pump 12 in place within an implant incision as described further below.

Pull tool 20 preferably comprises an elongated resilient section 44 having a hook 46 adjacent a first end 48 and a handle 50 adjacent a second end 52. Hook 46 and elongated section 44 are preferably fabricated from resilient metal wire, and are structured and configured to slidably fit through the elongated bore 28 in retainer body 22. Hook 46 is structured and configured to releasibly engage or hold catheter section 14.

Catheter section 14 as shown in FIG. 1 and FIG. 2 is generally the end portion of an elongated flexible catheter (not shown) that is implanted within patient, with catheter section 14 being the portion of catheter which extends out of an implant incision (not shown) in the patient. Catheter section 14 comprises a slack or “service” portion of catheter which allows handling of pump 12 by a surgeon after connector 34 on pump 12 has been coupled to the end 16 of catheter section.

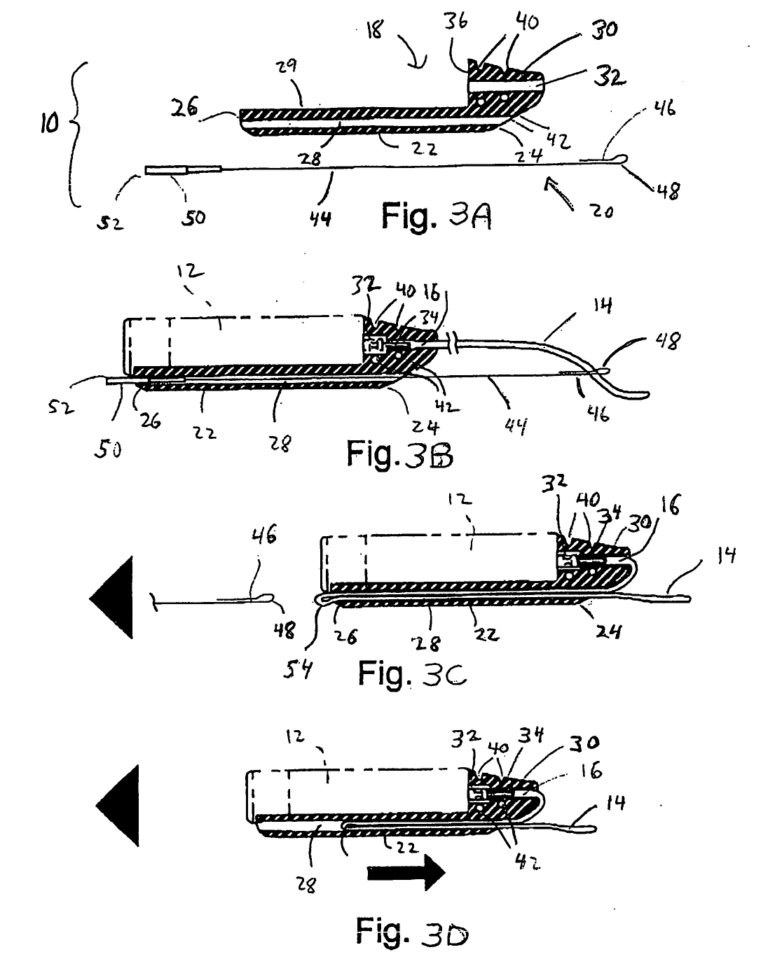

FIG. 3A through FIG. 3D illustrate generally the method of the invention. Prior to the implanting of pump 12, the retainer element 18 is coupled to pump 12. Catheter end 16 is passed through bore 32 in tip portion 30 and coupled or affixed to connector ferrule 24. Tip portion 30 is fitted over connector ferrule 34 on pump 12, with connector ferrule tensionally engaging the interior of bore 32 to hold retainer element 18 onto pump 12, with flat surface 36 or up portion 30 adjacent to the top end 38 (FIG. 2) of pump 12, and with arcuate surface 29 on retainer body 22 positioned adjacent the cylindrical exterior of pump 12. A biocompatible adhesive may be used to further secure retainer element 18 to pump 12. The slack or excess catheter portion 14 allows pump 12 and the attached retainer element 18 to be manipulated by a surgeon during the implant procedure.

Hook 46 and elongated section 44 of pull tool 20 are inserted into channel 28 in retainer body 22 at second end 26 and are passed through the elongated channel 28 as shown in FIG. 3B so that hook 46 extends out of channel 28 at first end 24. Catheter section 14 is then engaged by hook 46, and hook 46, together with the engaged catheter section 14, are drawn back into channel 28 in retainer body 22 as shown in FIG. 3C, with catheter section 14 forming a loop 54 as it is pulled through channel 28 by hook 46. Once loop 54 has been drawn or pulled through channel 28 to second end 26, hook 46 is disengaged from catheter section 14 to leave the looped catheter section 14 within channel 28. At this point the pump 12 and attached retainer element 18 may be sutured in place at a desired location within the implant incision utilizing suture grooves 40 and holes 42 (sutures are not shown), after which the implant incision is closed. The looped catheter section 14 is retained within channel 28 in retainer element 14 adjacent to pump 12 for the duration of implantation. The looped catheter section 14 thus is protected from unwanted fibrotic encapsulation by tissue surrounding the implanted pump, which would otherwise occur if catheter section 14 were left free or unmanaged within the implant incision.

The drug reservoir (not shown) internal to pump 12 will ultimately become exhausted or depleted during the implant period, typically after several weeks or months, and pump 12 will require replacement. A new incision (not shown) then is made to remove the depleted pump 12. Since the excess catheter portion 14 is retained or “managed” within the retainer element 18, there is no risk of cutting the catheter portion 14 with a scalpel when the new incision is made. The sutures holding the pump 12 and retainer element 18 in place are removed, and pump 12 and the attached retainer element 18 are withdrawn from the incision. As pump 12 and retainer element 18 are withdrawn, the looped catheter section 14 is withdrawn from channel 28 in retainer body 22 as shown in FIG. 3D to again provide a service portion of catheter to facilitate handling of pump 12 by a surgeon. The retention of the catheter section 14 within retainer element 18 during the implant avoids fibrotic encapsulation of catheter section 14 as noted above, and catheter section 14 is thus immediately available as a service loop without requiring any additional procedure to remove catheter section 14 from surrounding tissue. Catheter end 16 is detached from connector ferrule 34 to allow attachment to a replacement pump, which is then implanted by generally repeating the above procedure. The same retainer element 18 may be used with the replacement pump, or the retainer element 18 may be disposed with the spent pump.

Accordingly, it will be seen that this invention provides a catheter loop manage apparatus and method which controls the slack portion of a catheter located adjacent to an implanted pump and prevents fibrotic encapsulation of the slack catheter portion which could otherwise lead to damage to the catheter portion or complication of the implant procedure. Although the description above contains many specificities, these should not be construed as limiting the scope of the invention but as merely providing an illustration of the presently preferred embodiment of the invention. Thus the scope of this invention should be determined by the appended claims and their legal equivalents.

What is claimed is:

1. A catheter loop manager comprising a retainer element configured to associate with an implantable pump and to releasibly hold a loop of catheter.

2. The catheter loop manager of claim 1, wherein said retainer element is configured to coupled to said implantable pump and to hold said loop of said catheter adjacent to said implantable pump.

3. The catheter loop manager of claim 1, wherein said retainer element comprises a retainer body and a tip portion coupled to a front end thereof, said tip portion configured to engage a catheter connector on said implantable pump, said retainer body configured to hold said loop of said catheter.

4. The catheter loop manager of claim 3, wherein said retainer body includes an internal channel, said channel configured to hold said loop of said catheter therein.

5. The catheter loop manager of claim 4, further comprising a pull tool, said pull tool having a hook configured to fit through said channel and releasibly engage said catheter.

6. A catheter loop manager apparatus comprising a retainer element configured to couple to an implantable pump and releasibly hold a loop of catheter adjacent to said implantable pump.

7. The catheter loop manager apparatus of claim 6, wherein said retainer element includes an elongated retainer body having an elongated channel extending therethrough, said channel configured to hold said loop of catheter.

8. The catheter loop manager apparatus of claim 7, wherein said retainer element further comprises a tip portion joined to a first end of said elongated retainer body, said tip portion configured to engage a catheter connector on said implantable pump.

9. The catheter loop manager apparatus of claim 8, wherein said tip portion includes a bore, said bore configured to fit over said catheter connector and allow an end of said catheter to couple to said connector.

10. The catheter loop manager apparatus of claim 7, further comprising a pull tool, said pull tool having a hook configured to pass through said channel and releasibly engage said catheter.

11. The catheter loop manager apparatus of claim 6, wherein said retainer element includes at least one lateral groove configured to accommodate a suture.

12. A catheter loop manager apparatus usable with an implantable pump, said catheter loop manager apparatus comprising:
(a) a retainer element having an elongated channel extending therethrough said channel configured to retain a loop of catheter; and
(b) a pull tool having a hook configured to fit through said elongated channel and draw said loop of said catheter into said channel.

13. The catheter loop manager apparatus of claim 12, wherein said retainer element is configured to coupled to said implantable pump.

14. The catheter loop manager apparatus of claim 13, wherein said retainer element comprises an elongated retainer body and a tip portion joined to said elongated retainer body, said channel extending through said elongated retainer body, said tip portion configured to fit over a catheter connector on said implantable pump.

15. The catheter loop manager apparatus of claim 14, wherein said elongated retainer body includes an arcuate surface configured to conform to said implantable pump.

16. The catheter loop manager apparatus of claim 13, wherein said tip portion includes a bore configured to fit over said catheter connector and allow a catheter end to affix to said catheter connector.

17. The catheter loop manager apparatus of claim 12, wherein said retainer element includes at least one lateral groove configured to accommodate a suture.

18. A method for managing a service loop of catheter in association with an implantable pump, comprising:
(a) providing a retainer element, (b) affixing a catheter end to a connector on said pump; and
(c) drawing an excess portion of catheter into said retainer element to form a catheter loop therein.

19. The method of claim 18, further comprising coupling said retainer element to said pump.

20. The method of claim 18, further comprising coupling withdrawing said catheter loop from said retainer element after depletion of said implantable pump.

21. The method of claim 19, further comprising suturing said retainer element and said implantable pump within an implant incision.

22. The method of claim 18, wherein said retainer element includes a channel extending therethrough and said drawing is carried out with a pull tool having a hook configured to pass through said channel and engage said excess portion of catheter.
